(19) United States
(12) Patent Application Publication
(10) Pub. No.: US 2009/0076420 A1
(43) Pub. Date: Mar. 19, 2009

(54) METHOD AND DEVICE FOR ADIPOSE TISSUE TREATMENT

(75) Inventor: Michael KREINDEL, Haifa (IL)

Correspondence Address: BROWDY AND NEIMARK, P.L.L.C.;624 NINTH STREET, NW, SUITE 300, WASHINGTON, DC, 20001-5303, US

(73) Assignee: SYNERON MEDICAL LTD., Yokneam IIIit (IL)

(21) Appl. No.: 12/325,025

(22) Filed: Nov. 28, 2008

Related U.S. Application Data
(62) Division of application No. 10/421,938, filed on Apr. 24, 2003, now abandoned.

(51) Int. Cl. A61N 7/00

(52) U.S. Cl. 601/2

(57) ABSTRACT

A system and method for treatment of adipose tissue. One or more shock waves are generated focused on a region of adipose tissue located between 0.2 and 3 cm below the skin surface.

### FIELD OF THE INVENTION

The invention relates to medical devices and more specifically to such devices for the destruction of adipose tissue.

### BACKGROUND OF THE INVENTION

Various types of devices have been used for treatment of adipose tissue. Most of these methods rely upon the fact that adipose tissue is less durable mechanically than other body tissues such as skin and muscles. The problem is that adipose tissue is located under the skin layer, and energy applied to degrade adipose tissue should preferably reach the adipose tissue without damaging the skin.

One popular method of fat treatment is liposuction, which is based on mechanical disruption of fat with subsequent suction of the resulting debris out of the body. The main disadvantage of this method is its invasive character.

U.S. Pat. No. 5,143,063 describes a method treating adipose tissue based on thermal destruction of fat by exposing adipose tissue to focused microwave or ultrasound waves. The intensity of the energy is determined so as to selectively destroy fat cells without damaging the skin or deep tissues.

U.S. Pat. No. 6,450,972 discloses a device for ultrasound irradiation of adipose tissue in which the ultrasound waves are not focused, but the intensity of the waves is chosen for selective lipolysis.

U.S. Pat. No. 5,725,482 discloses superposition of ultrasound waves from two or more sources to create a wave having high intensity localized at the adipose tissue to be treated.

U.S. Pat. No. 6,500,141 improves treatment safety with ultrasound by forming the skin surface using suction.

U.S. Pat. No. 4,958,639 discloses destruction of calculi in the kidney using shock waves.

### SUMMARY OF THE INVENTION

The present invention provides a method and apparatus for the treatment of adipose tissue. In accordance with the invention, sonic shock waves are created and focused on a region of subcutaneous fat to be treated. The energy of the shock wave is selected to selectively destroy fat cells without damaging adjacent connective tissue or blood vessels.

Any method for generating shock waves and focusing the waves may be used in accordance with the invention. The focal point should preferably be at a depth of 0.5 to 3 cm below the skin surface, which is the typical depth of subcutaneous the fat layer.

In one embodiment of the invention, a shock wave is created by an electrical discharge through a medium. The medium may be a liquid such as water or a dielectric solid. The wave is focused onto a region of adipose tissue to be treated using a reflector having different acoustic properties than the medium. Other methods for creating shock waves include, but are not limited to, micro-explosions generated by a pulsed focused high intensity laser beam in a gas or liquid, a high rising magnetic field, or by piezoelectric transducers.

Typical structure of a shock wave is shown in FIG. 3. While not wishing to be bound by a particular theory, it is believed that at the shock front 301, a high pressure compresses the fat cells causing their destruction. This is followed by a negative pressure phase 302 that creates cavitation in the intracellular liquid causing an additional disruptive effect on the cells. As explained below, the energy of the shock wave is optimized for selective damage of adipose tissue without damaging adjacent blood vessels and connective tissue.

Thus, in one of its aspects, the invention provides a system for treatment of adipose tissue having an applicator comprising;
(a) A housing containing a medium;
(b) one or more shock wave generators configured to generate a shock wave in the medium; and
(c) One or more elements configured to focus a shock wave in the medium on a focal point located outside the housing at a distance of between 0.2 and 3 cm away from a surface of the housing.
   In its second aspect, the invention provides a method for treatment of adipose tissue comprising;
(a) Generating one or more shock waves in a medium and
(b) focusing one or more of the shock waves on a region of the adipose tissue located between 0.2 and 3 cm below the skin surface.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for treatment of adipose tissue using focused shock wave in accordance with the invention;

FIG. 2 shows an applicator for on adipose tissue treatment using focusing shock wave in accordance with the invention; and

FIG. 3 shows pressure behavior around a shock wave;

### DETAILED DESCRIPTION OF PREFERRED EMBODIMENTS

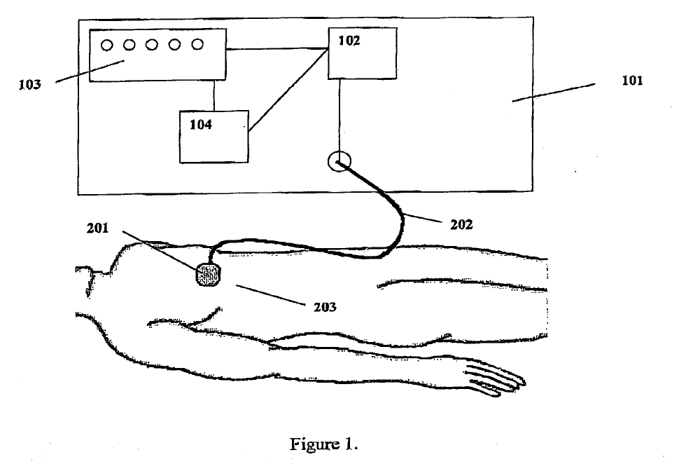

FIG. 1 shows a device for applying essentially focused shock waves to adipose tissue, in accordance with the invention. An applicator 201, to be described in detail below, contains a source of acoustic waves. The applicator 201 is adapted to be applied to the skin of an individual 203 in the treated region. The applicator 201 is connected to a control unit 101 via a harness 202. The control unit 101 includes a power source 102 and control panel 103. The power source 102 generates an electrical pulse in the applicator 201 via wires in the harness 202. The control unit 101 has an input device such as the control panel 103 that allows an operator to input selected values of parameters of the treatment such as the shock wave intensity. The control unit 101 optionally contains a processor 104 for monitoring and controlling various functions of the device.

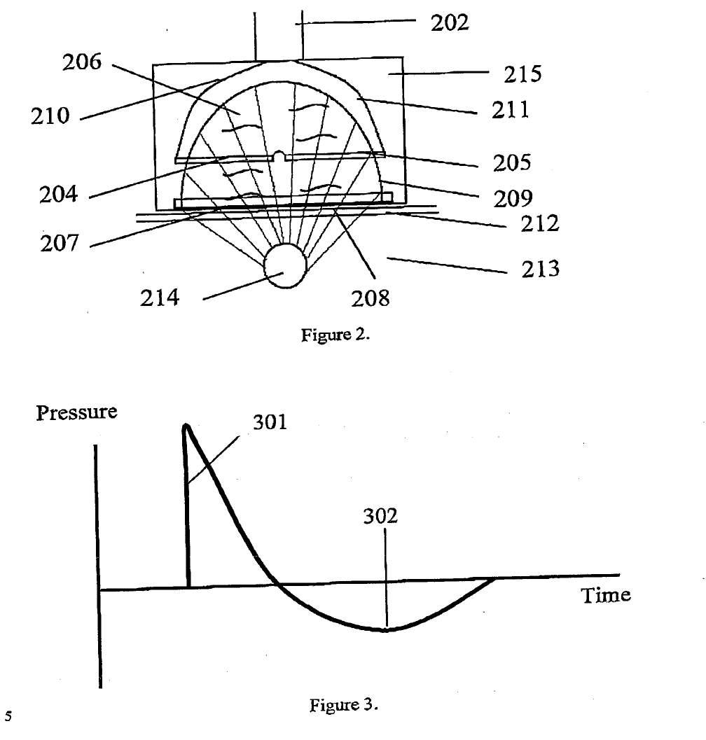

FIG. 2 shows one embodiment of the applicator 201 in detail. The applicator 201 has a housing 215. The interior of the housing 215 is filled with a medium 206. A pair of electrodes 204 and 205 extend into the medium 206 from the housing 215 that are separated by a gap 203. The electrodes 204 and 205 are connected electrically through wires 210 and 211 that extend along the harness 202 to the control unit 101. When a voltage pulse is applied across the electrodes 204 and 205 by the power supply 102, an electrical discharge 205 is generated in the gap 203 is through the medium 206. The electrical discharge 205 generates a sonic shock wave in the medium 206.

A metal reflector 209 lines the inside surface of the housing 215. The shock wave originating at the gap 203 propagates away from the gap 203 in all directions through the medium 206. The housing 215 is shaped such that the reflector 209 reflects the sonic wave and focuses it outside the housing 215 on a region 214 to be treated in the fat layer 213. A plastic output window 207 having acoustic properties similar to that of body tissues provides an interface between applicator 201 and the region to be treated 214. The focal point is 0.2 to 3 cm beyond the window 207, so that when the window 207 is applied to the skin surface, the sonic waves are focused at a depth of 0.2 to 3 cm below the skin surface, which coincides with the depth of the subcutaneous adipose tissue. A medium 208 such as a water-based gel such as Vaseline may be use for acoustic coupling between the applicator 201 and the skin 212.

For example, the housing 215 may have a truncated ellipsoidal shape, as shown in FIG. 2. The gap 203 is located at one focus of the ellipsoid, and when the applicator is applied to the skin 212, the region to be treated 214 is located at the other focus. Thus, a shock wave in the liquid 206 originating at the gap 203 will be reflected by the reflector 209 to the region to be treated 214.

The efficacy of the shock wave treatment may be enhanced by shaping the skin so as to bring the region to be treated 214 closer to the surface. For example, suction may be applied to the skin surface over the region 214 in order cause the skin to form a mound including the region 214.

The length of the gap 203 (i.e. the distance between electrode tips) is preferably not larger than 5 mm. Each shock wave should preferably have an energy between to 0.1 to 2 Joules in order to avoid damage to the skin surface. A sequence of pulse waves having a total energy exceeding 50 Joules should be delivered to the treated zone.

1. A system for treatment of adipose tissue having an applicator comprising;
(a) A housing containing a medium;
(b) one or more shock wave generators configured to generate a shock wave in the medium; and
(c) One or more elements configured to focus a shock wave in the medium on a focal point located outside the housing at a distance of between 0.2 and 3 cm away from a surface of the housing.

2. The system according to claim 1 wherein the energy of a shock wave is 0.1 to 2 Joules.

3. The system according to claim 1 configured to deliver a sequence of shock waves.

4. The system according to claim 3 wherein a sequence of shock waves delivers an energy of 50 Joules to the adipose tissue.

5. The system according to claim 1 further comprising a processor configured to determine one or more parameters of the shock wave.

6. The system according to claim 1, wherein a shock wave generator comprises a pair of electrodes separated by a gap in the medium, and wherein a shock wave is generated by an electrical discharge in the gap.

7. The system according to claim 1 wherein the medium is water.

8. The system according to claim. 1 wherein the medium is a dielectric solid.

9. The system according to claim 6 wherein an element is a reflector having a truncated ellipsoidal shape, and the gap is located at a focus of the ellipsoid.

10. The system according to claim 6 wherein the electrodes are bipolar.

11. The system according to claim 1 where a shock wave generator comprises one or more piezoelectric transducers.

12. The system according to claim 1 wherein the shock wave is generated by a pulsed focused laser beam in a gas or liquid.

13. The system according to claim 1 wherein the shock wave is generated by a high rising magnetic field.

14. The system according to claim 1 further comprising means for shaping skin for optimal shock wave delivery.

15. The system according to claim 9 wherein the means for shaping skin is suction.

16. A method for treatment of adipose tissue comprising;
(a) Generating one or more shock waves in a medium and
(b) focusing one or more of the shock waves on a region of the adipose tissue located between 0.2 and 3 cm below the skin surface.

17. The method according to claim 16 wherein the energy of a shock wave is 0.1 to 2 Joules.

18. The method according to claim 16 comprising delivering a sequence of shock waves to the adipose tissue.

19. The method according to claim 18 wherein a sequence of shock waves delivers an energy of 50 Joules to the adipose tissue.

20. The method according to claim 16, wherein a shock wave is generated by an electrical discharge in a gap in the medium separating a pair of electrodes.

21. The method according to claim 16 wherein the medium is water.

22. The method according to Claim 16 wherein the medium is a dielectric solid.

23. The method according to claim 16 wherein a shock wave is focused by a reflector having an ellipsoidal shape, and the gap is located at a focus of the ellipsoid.

24. The method according to claim 19 wherein the electrodes are bipolar.

25. The method according to claim 16 where a shock wave generator comprises one or more piezoelectric transducers.

26. The method according to claim 16 where the shock wave is generated by a pulsed focused laser beam in a gas or liquid.

27. The method according to claim 16 wherein the shock wave is generated by a high rising; magnetic field.

28. The method according to claim 16 further comprising shaping skin for optimal shock wave delivery.

29. The method according to claim 23 wherein the skin is shaped by suction.
